(19) United States
(12) Patent Application Publication
(10) Pub. No.: US 2022/0184366 A1
(43) Pub. Date: Jun. 16, 2022

(54) VASCULAR ACCESS SHEATH AND CATHETER SYSTEM

(71) Applicant: Interventional Technological LLC, Tucson, AZ (US)

(72) Inventors: Robert E. Spanos, JR., Longboat Key, FL (US); John Paul Pacanowski, Tucson, AZ (US)

(21) Appl. No.: 17/548,763

(22) Filed: Dec. 13, 2021

Related U.S. Application Data
(60) Provisional application No. 63/124,169, filed on Dec. 11, 2020.

(51) Int. Cl. A61M 39/02; A61M 29/00; A61M 39/06; A61M 25/01; A61M 25/00; A61M 25/09

(52) U.S. Cl. CPC A61M 39/0247; A61M 29/00; A61M 39/06; A61M 25/0108; A61M 2039/0279; A61M 25/0032; A61M 25/09; A61M 2039/0258; A61M 25/005

(57) ABSTRACT

A vascular access sheath includes an inner support structure extends from a hemostasis valve toward a distal end section of the access sheath. An outer polymer is disposed over the inner support structure, where the outer polymer has an exterior surface extending from the hemostasis valve to a distal tip of the access sheath. The outer polymer has a consistent thickness along the access sheath and defines a greater durometer an intermediate section of the access sheath than at the proximal and distal end sections of the access sheath. The outer polymer also has a secondary lumen disposed between the exterior surface and the central lumen, where the secondary lumen extends at least partially along the length of the access sheath and is configured to receive a wire to reinforce the stiffness of the intermediate section of the access sheath.

### CROSS REFERENCE TO RELATED APPLICATION

This application claims priority from U.S. Provisional Application No. 63/124,169, filed Dec. 11, 2020, the entire contents of which are incorporated by reference.

### TECHNICAL FIELD

This application relates to catheter systems including an access sheath and methods of using such access sheathes for intravascular medical procedures.

### BACKGROUND

Intravascular catheters are commonly used as an effective method for treating various types of vascular disease. Peripheral vascular intervention procedures traditionally utilize intravascular catheters that position an access sheath within a patient's vasculature so that various devices and instruments can be inserted through the access sheath to a targeted operating site. For example, procedures that utilize an access sheath include angioplasty, chronic total occlusion (CTO) cross procedures, procedures to treat peripheral arterial disease, procedures to treat infrainguinal arterial disease, and the like.

When performing intravascular procedures, access sheaths are typically inserted into a vessel using a flexible interior dilator that is advanced along a guide wire to the operating site. Catheters that are advanced over a guide wire can be referred to as an over-the-wire (OTW) catheter. A guide wire is inserted into the patient's vasculature at a suitable location through a percutaneous access, such as a contralateral femoral access, pedal arterial access, or radial arterial access. The guide wire is then advanced to or near the targeted intravascular operating site, such as with the assistance of radiographic imaging.

With a guide wire in place, access sheathes are commonly advanced over the guide wire assembled with the interior dilator. For procedures involving contralateral femoral access, access sheaths are typically advanced across the aortic bifurcation and extend to the operating site, such as in the common femoral artery or in the ipsilateral leg arteries. Once a traditional access sheath is positioned with an assembled dilator in the desired location in the vasculature, the dilator is then usually withdrawn from the interior of the access sheath.

In procedures where a traditional access sheath has assumed a relatively tight radiused bend or angular transition in the vasculature, such as when transitioning the aortic bifurcation, there is a substantial risk that the access sheath can kink at the tight bend, such as when the dilator is removed. A kink in an access sheath can severely complicate and slow the procedure. For example, devices and instruments can be difficult to transition through a kinked area of a sheath and in some instances may be prohibited from advancing through the kinked area or areas.

However, presently known measures to prevent or reduce kinking of intravascular access sheaths focus on kink prevention near the access site or as a result of torque or navigational manipulation of a sheath by the surgeon at the proximal end. For example, it is known that the wall thickness of an access sheath can be increased at the proximal end, such as by increasing the thickness of an outer polymer that is usually disposed over an interior reinforcement coil. Yet, the increased thickness of the sheath results in a larger outer diameter of the sheath which causes other serious and life-threatening complications for the patient, such as providing a higher potential of creating issues at the access site due the resulting increase to the arteriotomy size, such as issues like bleeding, hematoma, pseudoaneurym, and embolism. Likewise, increasing wall thickness that results in a smaller inner diameter of an access sheath is undesirable because it reduces the size limitations of medical devices that can be used with such access sheaths.

### SUMMARY

These and other needs are met by the present disclosure, which presents a vascular catheter system that has an access sheath and a corresponding method of treatment, such as a method for treating contralateral lower extremity vascular condition using the access sheath. The access sheath has a reinforced intermediate section to be positioned at tortuous curvatures of vascular anatomy, such as the aortic bifurcation, so as to reduce the risk of kinking, such as when an interior dilator is removed for operating within the access sheath. The reinforced intermediate section may be provided by one or a combination of reinforcement features, including an increased durometer of the outer polymer of the access sheath and a secondary or auxiliary wire inserted along a secondary or auxiliary lumen disposed in the wall of the access sheath.

In some implementations, the combination of the increased polymer durometer and an auxiliary wire in the same access sheath may provide the benefits of both features individually, as well as provide a synergistic effect. For example, the integration of the increased durometer and the auxiliary wire can provide a central access lumen that decreases the duration of the medical procedure and reduce the occurrence of complications. In other examples, the access sheath can provide a consistent and manageable outer diameter at the access site and have a relatively lower durometer at the end sections of the access sheath to allow for greater ease of accurately inserting and position of the access sheath. Further, in some examples, the auxiliary wire may also be used for anchoring the access sheath near a target site or treatment location, which may ensure that the devices and instruments using the access sheath are properly positioned when exiting the sheath.

In some aspects, the vascular catheter system comprises a flexible dilator and an access sheath. The flexible dilator has an interior channel that slidably engages an intravascular guide wire that leads toward a target treatment location in a patient's anatomy. The access sheath has a tubular shape that defines a central lumen and is disposed over the flexible dilator so as to simultaneously advance with the flexible dilator along the intravascular guide wire toward the target treatment location. The flexible dilator is configured to be withdrawn from the central lumen of the access sheath to provide tool access to a target treatment location. The access sheath comprises an inner support structure and an outer polymer. The inner support structure is disposed around the central lumen and extends at least partially along a length of the access sheath. A radiopaque marker is disposed at a distal end section of the access sheath, and a hemostasis valve disposed at a proximal end section of the access sheath. The outer polymer encases the inner support structure and the radiopaque marker, such that the exterior surface of the outer polymer extends between the radiopaque marker and the hemostasis valve to define a consistent outer diameter. The outer polymer includes a first durometer at the proximal end section of the access sheath, a second durometer at an intermediate section of the access sheath, and a third durometer at the proximal end section of the access sheath, where the second durometer is greater than the first and third durometers. When the flexible dilator is removed from the central lumen of the access sheath, the second durometer of the outer polymer is configured to prevent the intermediate section of the access sheath from collapsing or kinking.

In some aspects, the outer polymer includes a secondary lumen disposed between the exterior surface and the central lumen, where the secondary lumen extends at least partially along the length of the access sheath. In some examples, the secondary lumen has an exit opening at the exterior surface of the outer polymer at a longitudinal location proximal to a distal tip of the access sheath. Further, in some examples, the radiopaque marker is disposed at or near the exit opening for positioning the exit opening at a desired location in a patient's anatomy. A secondary wire is configured to be inserted along the secondary lumen to the exit opening, such that when the flexible dilator is removed from the central lumen of the access sheath, the secondary wire is configured to prevent the intermediate section of the access sheath from kinking. In some examples, the secondary lumen may also or alternatively be used to dispense drugs and/or contrast agents through the lumen.

In some aspects, the secondary wire has a blunted tip and a hydrophilic coating that is configured to reduce friction when advancing through the secondary lumen. Also, in some examples, the secondary lumen has an entrance opening that is engaged with a valve port of the hemostasis valve. The valve port may thus be configured to receive the secondary wire via an introducer device and direct the secondary wire into the secondary lumen.

The secondary lumen, in some implementations, assumes a contracted state when the secondary wire is not present in the secondary lumen and an expanded state when the secondary wire is present in the secondary lumen, causing it to expand in diameter from an initial diameter in the contracted state. In some examples, with the secondary lumen in the expanded state, the exterior surface of the access sheath includes a longitudinal ridge defined by the expanded secondary lumen.

In some aspects, the inner support structure of the access sheath has a coil or a braiding with longitudinal flexibility. For example, the inner support structure may include a stainless steel coil or a stainless steel braiding. In some aspects, the outer polymer comprises polyether block amide (e.g., PEBAX®) formed as a single integral piece along the length of the access sheath. In some examples, the access sheath may also have an inner polymer that defines a lubricious inner surface of the central lumen. The inner polymer in some implementations also includes polyether block amide formed as a single integral piece with the outer polymer along the length of the access sheath. The wall thickness of the access sheath may be defined between the inner surface and the outer surface, and in some examples, the wall thickness is substantially consistent along the length of the access sheath.

Another aspect is a vascular access sheath that includes a hemostasis valve disposed at a proximal end section the access sheath. An inner support structure extends from the hemostasis valve toward a distal end section of the access sheath, where the inner support structure defines a tubular shape that extends around a central lumen of the access sheath. An outer polymer is disposed over the inner support structure, where the outer polymer has an exterior surface extending from the hemostasis valve to a distal tip of the access sheath. The outer polymer has a consistent thickness along the access sheath and defines a greater durometer an intermediate section of the access sheath than at the proximal and distal end sections of the access sheath. The outer polymer also has a secondary lumen disposed between the exterior surface and the central lumen, where the secondary lumen extends at least partially along the length of the access sheath and is configured to receive a wire to reinforce the stiffness of the intermediate section of the access sheath.

In some aspects, a radiopaque marker is disposed at the distal end section of the access sheath. In some examples, at least two radiopaque markers may be provided, such as a first radiopaque marker disposed at the distal tip the access sheath and a second radiopaque marker disposed at or near an exit opening of the secondary lumen. The exit opening may be at the exterior surface of the outer polymer at a location proximal to the distal tip of the access sheath, such as between 2 and 4 cm from the distal tip of the access sheath. In some implementations, an entrance opening of the secondary lumen is engaged with a valve port of the hemostasis valve, such that the valve port can receive the secondary wire and direct the secondary wire into the secondary lumen.

In some aspects, the secondary lumen has a contracted state defined by the secondary wire not being present in the secondary lumen and an expanded state defined by the secondary wire being present in and expanding the secondary lumen to a second diameter that is greater than the initial diameter in the contracted state. In some examples, the secondary lumen in the contracted state provides a cross-sectional shape that is a slotted shape, an ovular shape, a circular shape, or a crescent shape.

Another aspect is a method for treatment that initially involves inserting a guide wire is through the patient's skin and into a vascular lumen, such as a femoral, radial, or brachial artery and steered near a target site. For example, treating contralateral lower extremity vascular condition may involve advancing an intravascular guide wire from a contralateral percutaneous femoral access through an aortic bifurcation and into a common femoral artery (CFA). A catheter assembly is advanced over the guide wire, such as through the aortic bifurcation and into the CFA toward a target site or treatment location. The catheter assembly includes a flexible dilator having an interior channel that surrounds the guide wire and an access sheath disposed over the flexible dilator so as to simultaneously advance with the flexible dilator along the intravascular guide wire. The access sheath includes an inner support structure extending at least partially along a length of the access sheath and an outer polymer encasing the inner support structure and defining an exterior surface that extends from a distal tip of the access sheath to a proximal end section of the access sheath. The flexible dilator is withdrawn from the access sheath to expose a central lumen of the access sheath configured for a tool to access the target treatment location. When the flexible dilator is removed from the central lumen of the access sheath, an intermediate section of the access sheath spanning the aortic bifurcation is prevented from collapsing or kinking because a durometer of the outer polymer at the intermediate section exceeds a threshold hardness. The proximal end section and distal end section of the access sheath have durometers that are less than the durometer at the intermediate section to provide flexibility at the contralateral percutaneous femoral access and near the target treatment location.

In some aspects, prior to removing the flexible dilator from the access sheath, a secondary wire is inserted through a secondary lumen in the access sheath. The secondary lumen is disposed longitudinally within a thickness of the outer polymer and has an exit opening at the exterior surface of the outer polymer proximal to the distal tip. The secondary wire increases the stiffness of the access sheath to prevent the intermediate section of the access sheath from kinking. In some examples, the access sheath has a consistent diameter and wall thickness along the length of the access sheath. The secondary lumen, in some implementations, includes a contracted state that is defined by the secondary wire not being present in the secondary lumen and an expanded state defined by the secondary wire being present in and expanding the secondary lumen. With the secondary lumen in the expanded state, the exterior surface of the access sheath may include a longitudinal ridge defined by the expanded secondary lumen.

In some aspects, the access sheath is positioned in the vasculature using radiographic imaging to locate an exit opening of a secondary lumen in the access sheath near a desired anchoring anatomy. For example, the access sheath may have a radiopaque marker disposed at a distal end section of the access sheath near the exit opening, where the radiopaque marker is viewable on x-rays or other medical imaging devices. The secondary lumen is disposed longitudinally within a thickness of the outer polymer and has the exit opening at the exterior surface of the outer polymer proximal to the distal tip. Prior to removing the flexible dilator from the access sheath, a secondary wire may be inserted through the secondary lumen to the exit opening. Also, the secondary wire may be anchored at the desired anchoring anatomy for securing the access sheath near the target treatment location.

The details of one or more implementations of the disclosure are set forth in the accompanying drawings and the description below. Other aspects, advantages, purposes, and features will be apparent upon review of the following specification in conjunction with the drawings.

### BRIEF DESCRIPTION OF THE DRAWINGS

The following figures are provided by way of example and are not intended to limit the scope of the claimed invention.

FIG. 1 illustrates an exemplary human anatomy that shows portions of the vascular system and potential access site locations for a catheter system to enter a vessel.

FIG. 2A illustrates an access sheath in accordance with an aspect of the present invention that extends through a patient's skin at a contralateral percutaneous femoral access site and spans the aortic bifurcation to a target location in a common femoral artery (CFA).

FIG. 2B illustrates a prior access sheath with a kink formed in the aortic bifurcation.

FIG. 3A is a perspective view of a catheter system showing a flexible dilator and an access sheath in accordance with an aspect of the present invention.

FIG. 3B is a perspective view of a catheter system showing a flexible dilator and an access sheath in accordance with another aspect of the present invention.

FIG. 4A is a perspective view of a distal end section of the catheter systems of FIGS. 3A and 3B showing radiopaque markers and an end section of the inner coil disposed in the access sheath.

FIG. 4B is an exploded perspective view of the radiopaque markers and the distal end section of the inner coil shown in FIG. 4A.

FIG. 5A is a perspective view of a distal end section of the catheter systems of FIGS. 3A and 3B showing the access sheath disposed over the flexible dilator.

FIG. 5B is a cross-sectional perspective view of the access sheath and the flexible dilator taken at section VA shown in FIG. 5A.

FIG. 6A is a perspective view of a distal end section of the catheter systems of FIGS. 3A and 3B showing a secondary wire inserted in a secondary lumen of the access sheath.

FIG. 6B is a cross-sectional perspective view of the access sheath and the flexible dilator taken at section VIA shown in FIG. 6A.

FIG. 7A is a perspective view of a distal end section of the catheter systems of FIGS. 3A and 3B showing the flexible dilator and the access sheath over a guide wire.

FIG. 7B is a cross-sectional perspective view of the access sheath and the flexible dilator taken at section VIIA shown in FIG. 7A.

FIG. 8 is a side elevation view of components of a catheter system, showing a dilator, an inner coil and radiopaque markers, an access sheath, a secondary wire, and a guide wire.

FIG. 9A is a cross-sectional perspective view of a catheter system shown without a secondary wire or a guide wire.

FIG. 9B is a cross-sectional perspective view of the catheter system of FIG. 7A shown with the secondary wire extending through the secondary lumen.

FIG. 10A is a cross-sectional perspective view of a catheter system shown with a secondary wire outside of a secondary lumen.

FIG. 10B is an enlarged cross-sectional view of the catheter system taken at section XB shown in FIG. 10A, showing the secondary lumen without a secondary wire.

FIG. 10C is a cross-sectional perspective view of the catheter system of FIG. 10A shown with the secondary wire inserted in the secondary lumen.

FIG. 10D is an enlarged cross-sectional view of the catheter system taken at section XD shown in FIG. 10C, showing the secondary wire in the secondary lumen.

FIG. 11A is a cross-sectional perspective view of an additional example of a catheter system shown with a secondary wire outside of a secondary lumen.

FIG. 11B is an enlarged cross-sectional view of the catheter system taken at section XIB shown in FIG. 11A, showing the secondary lumen without a secondary wire.

FIG. 11C is a cross-sectional perspective view of the catheter system of FIG. 11A shown with the secondary wire inserted in the secondary lumen.

FIG. 11D is an enlarged cross-sectional view of the catheter system taken at section XID shown in FIG. 11C, showing the secondary wire in the secondary lumen.

FIG. 12A is a cross-sectional side view of the catheter system taken along plane XIIA shown in FIG. 5A, without a secondary wire.

FIG. 12B is an enlarged cross-sectional view of the catheter system taken at section XIIB shown in FIG. 12A and illustrating an exit opening of the secondary lumen at the exterior surface of the access sheath.

FIG. 13A is a cross-sectional side view of the catheter system taken along plane XIIA shown in FIG. 5A, with a secondary wire.

FIG. 13B is an enlarged cross-sectional view of the catheter system taken at section XIIIB shown in FIG. 13A and illustrating the secondary wire in the access sheath.

FIGS. 14A-14D are illustrations of examples of access sheaths with corresponding charts of durometer over the length of the respective access sheath.

FIG. 14E is an illustration of an example of an access sheath and a secondary wire with a corresponding chart of durometer of the access sheath and combined access sheath and secondary wire over the length of the respective access sheath.

FIG. 15 is an illustration of a catheter system initially being advanced over a guide wire that extends through a patient's skin at a contralateral percutaneous femoral access site and spans an aortic bifurcation to a target location in a common femoral artery (CFA).

FIG. 16 is an illustration of the catheter system shown in FIG. 15 advanced further over the guide wire partially over the aortic bifurcation.

FIG. 17 is an illustration of the catheter system shown in FIG. 16 advanced further over the guide wire toward the target location in the CFA and a secondary wire advanced through the access sheath to an anchoring location.

FIG. 18 is an illustration of the catheter system shown in FIG. 17 (having an added port and syringe) with the guide wire and the flexible dilator removed from the access sheath.

Like reference numerals in the various drawings indicate like elements.

### DETAILED DESCRIPTION

The present disclosure provides a vascular catheter system, an access sheath, and corresponding methods of using the catheter system and the access sheath during intravascular surgical procedures. Although certain embodiments and examples are described below, those skilled in the art will recognize that the inventive concepts extends beyond the specifically disclosed embodiments and/or uses and obvious modifications and equivalents thereof. Thus, it is intended that the scope of the inventive concepts presented herein should not be limited by any particular embodiments described below.

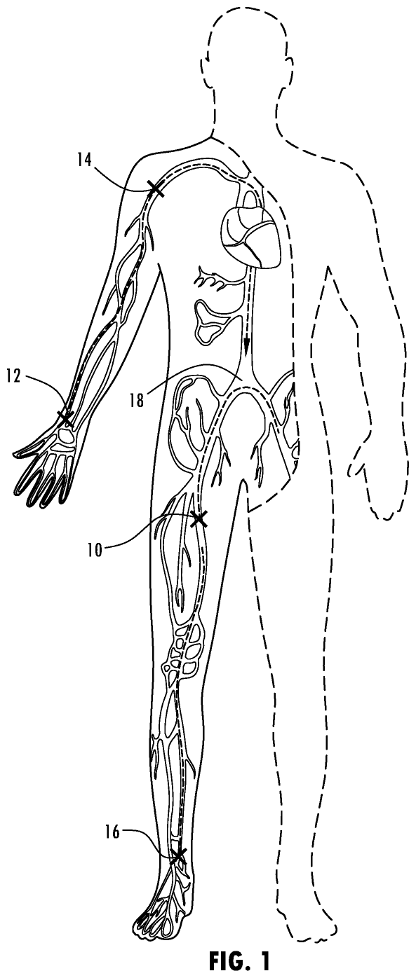

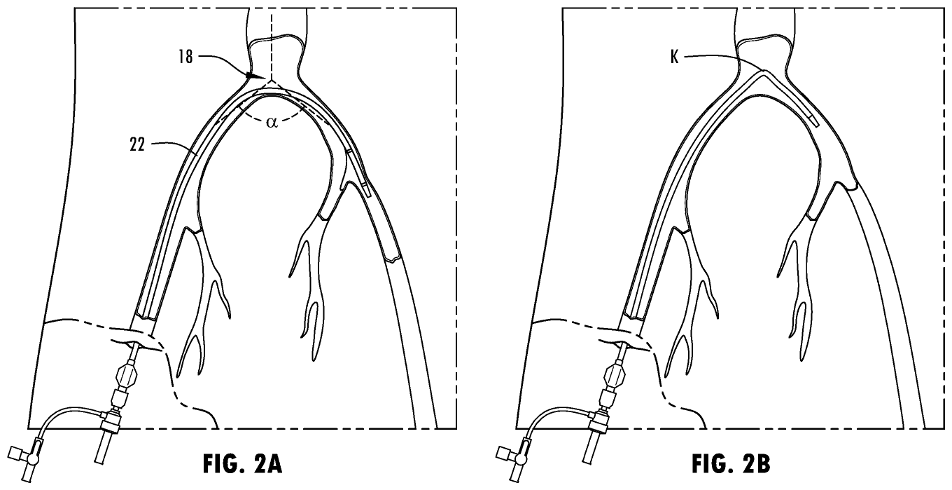

As shown in FIG. 1, an exemplary human anatomy illustrates portions of a vascular system and some potential percutaneous access site locations for a catheter system to access a vessel. As generally understood, the vascular anatomy has various tortuous curvatures. For example, a femoral access site 10 is shown for a potential contralateral femoral access with a catheter system that is advanced through the vasculature to span the aortic bifurcation 18 and extend toward a target location, such as in a common femoral artery (CFA) as shown in FIG. 2A. As used herein, the term “contralateral” means the side of the body opposite to the side that is the target of intervention, such as the right leg femoral access site 10 opposite the left leg target treatment location.

In addition, a radial arterial access site 12 and a transbrachial arterial access site 14 are shown in FIG. 1, either of which may advantageous for accessing certain target treatment locations in the vascular system, such as for various intravascular procedures. Further, a pedal arterial access site 16 is shown in FIG. 1, which similarly may be utilized in intravascular procedures for accessing a target treatment location in the vascular system. The catheter system and access sheath described herein may be configured for use with a particular access site and to advance through certain tortuous sections of the vasculature, such as those shown in FIG. 1 and described herein.

With reference to percutaneous femoral access for a contralateral retrograde femoral approach, the catheter system spans the aortic bifurcation 18, such as shown in FIG. 2A. The aortic bifurcation 18 is the vasculature section where the abdominal aorta bifurcates in to the left and right common iliac arteries. The common iliac arteries diverge as they descend and divide at the level of sacro-iliac joint into external and internal iliac arteries, including the common femoral artery. Although each individual patient has a relatively unique vascular anatomy, the aortic bifurcation commonly provides a relatively sharp or acute angle a (FIG. 2A) that is difficult to transition across with a catheter, such as, for example, an angle of between 30° and 70°, although other angles of the aortic bifurcation are common, such as less than 100°, approximately between 40° and 60°, approximately between 20° and 50°, approximately between 50° and 90°, and approximately between 20° and 90°. In addition to the angle presented at the aortic bifurcation, the distance at which the bifurcation transitions between the common iliac arteries is relatively short, generally occurring across the width of the abdominal aorta at the infrarenal level, such as approximately less than 30 mm or approximately between 25 mm and 15 mm. Accordingly, to advance and span the tight curvature of the aortic bifurcation, an intravascular catheter is commonly required to bend to the necessary angle, such as approximately 45° at a radius of approximately less than 15 mm.

Various method are known to transition an aortic bifurcation with an intravascular catheter, such as by deploying an access sheath along a guide wire with an internal dilator that occupies and supports the central lumen of the access sheath. Once such an access sheath is positioned across the aortic bifurcation, the internal dilator is removed for a surgeon to utilize the central lumen of the access sheath. However, as illustrated for reference in FIG. 2B, a prior access sheath that spans the aortic bifurcation may form a kink K at the aortic bifurcation when the internal dilator is removed. Such a kink in the access sheath at the aortic bifurcation may narrow and restrict the central lumen of the access sheath, which severely complicates use of the access sheath and the corresponding procedure.

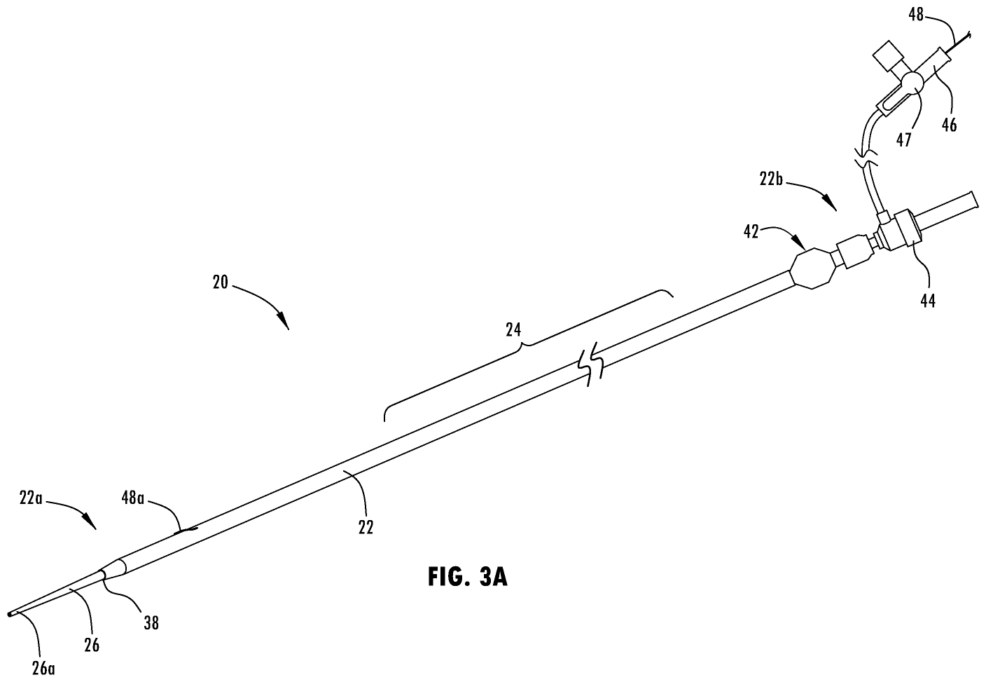

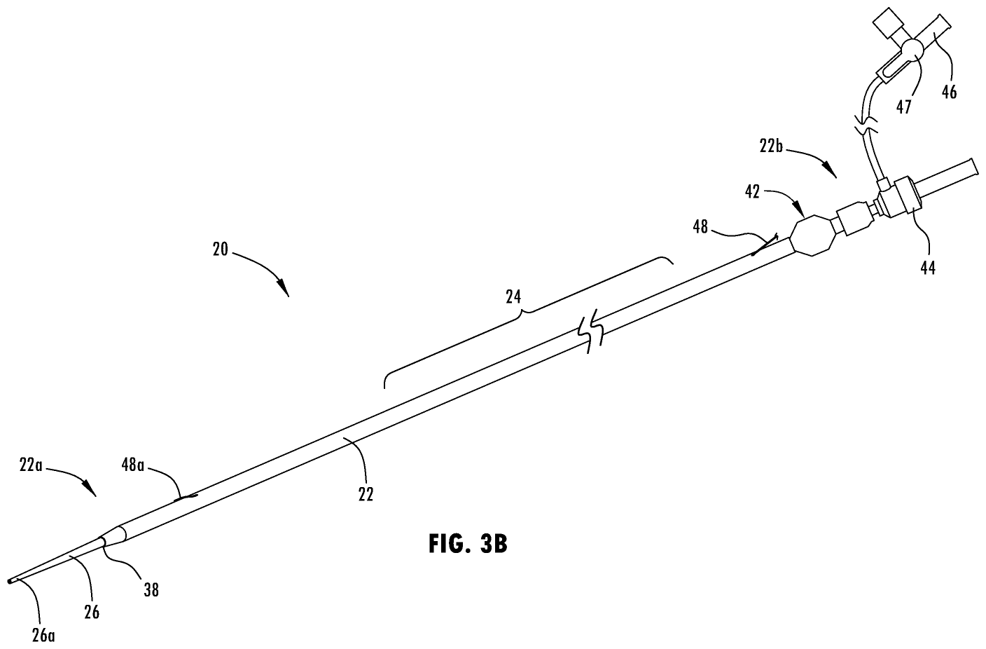

Referring now to FIGS. 3A and 3B, a vascular catheter system 20 includes an access sheath 22 that has a reinforced intermediate section 24 that is configured to be positioned at a tortuous curvature or curvatures of vascular anatomy, such as the aortic bifurcation 18 (FIG. 2A). The reinforced intermediate section 24 is configured to reduce the risk of the access sheath kinking, such as when an interior dilator 26 is removed for operating within the access sheath 22, such as shown in the example illustrated in FIG. 2A. The intermediate section of the access sheath is generally defined as a longitudinal section of the access sheath that is located between the distal and proximal end sections 22a, 22b, as described further herein. To provide anti-kinking properties, the intermediate section of the access sheath described herein may be provided with one or a combination of reinforcement features. For example, an increased durometer of the outer polymer of an access sheath may be incorporated with a secondary or auxiliary wire inserted along a secondary or auxiliary lumen disposed in the wall of the access sheath. In some implementations, the combination of the increased polymer durometer and an auxiliary wire in the same access sheath may provide the benefits of both features individually, as well as provide a synergistic effect.

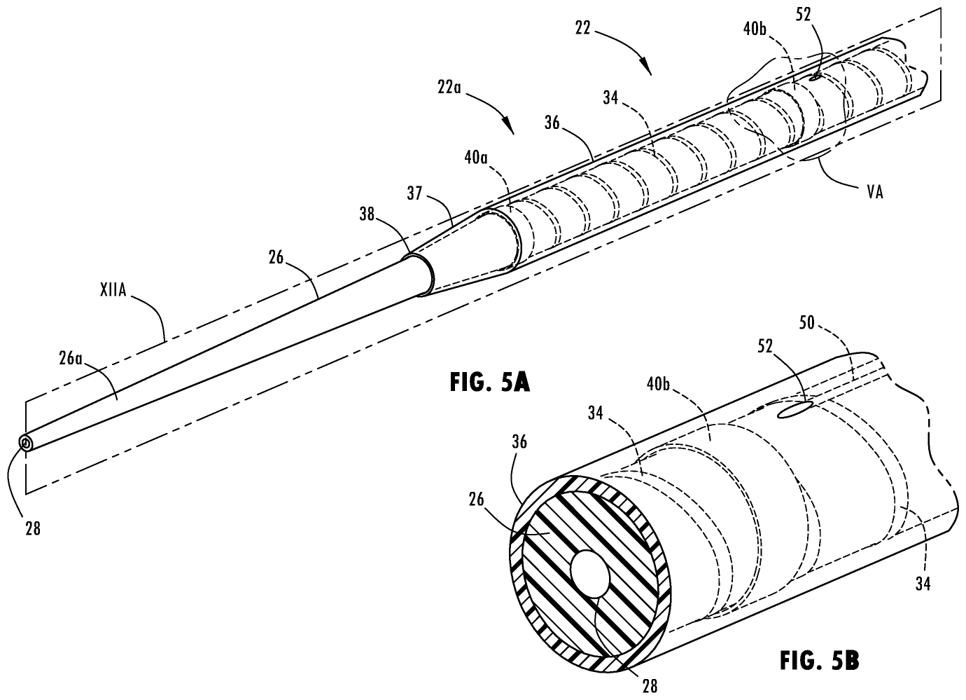

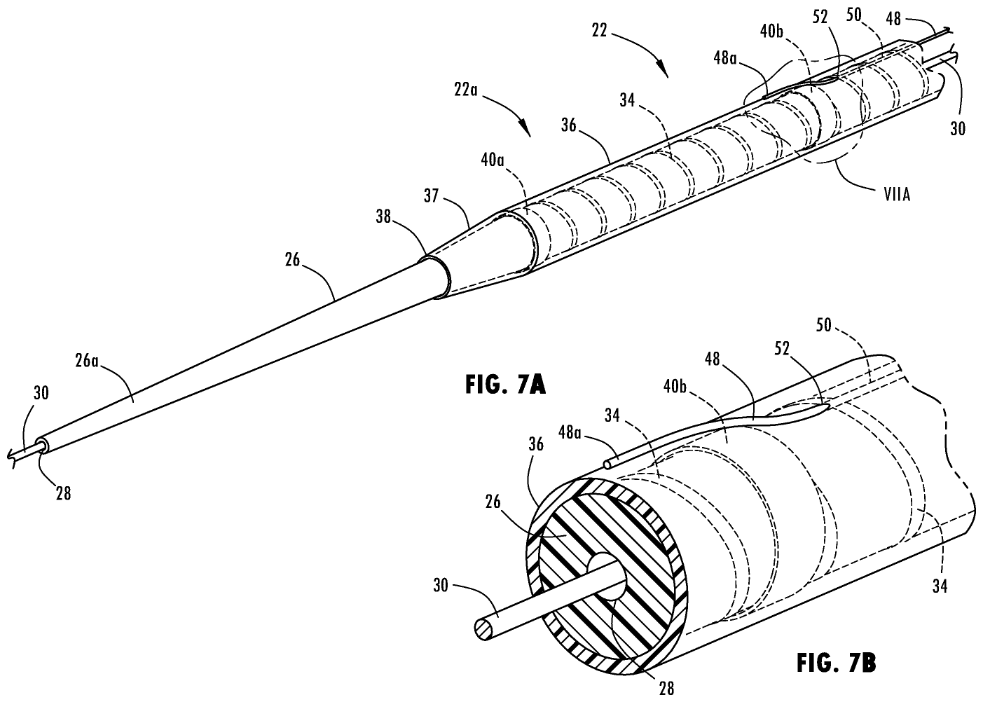

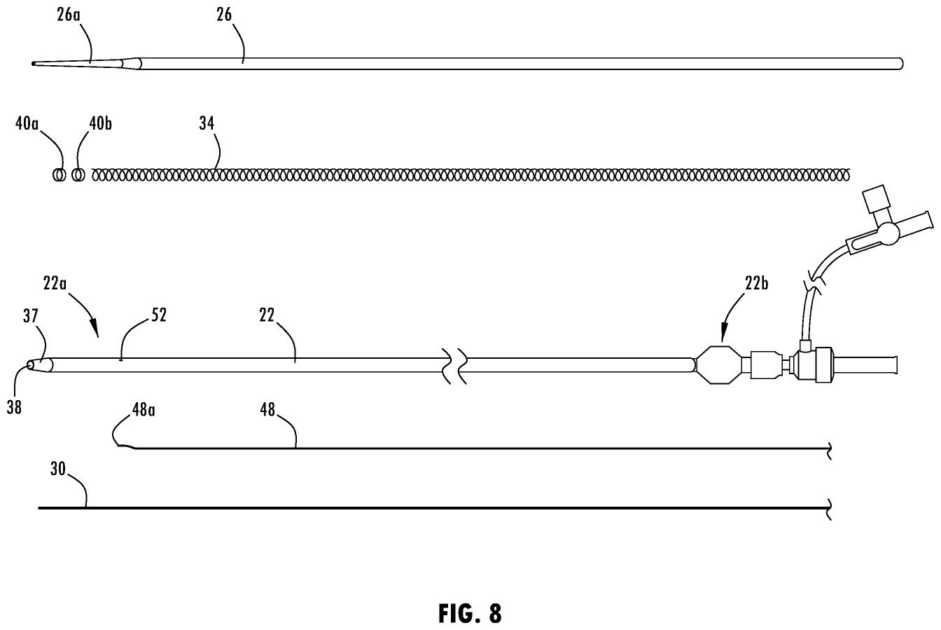

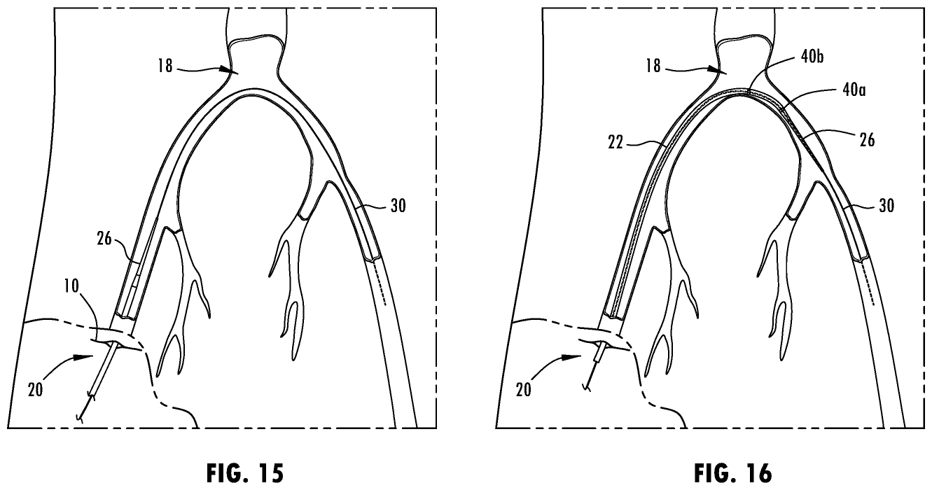

As show in FIGS. 3A and 3B, the vascular catheter system 20 comprises a flexible dilator 26 and an access sheath 22. The flexible dilator 26 has an interior channel 28 (FIG. 5A) that slidably and removably engages an intravascular guide wire 30 (e.g., a nitinol wire as shown in FIG. 7A) that leads toward a target treatment location in a patient's anatomy, such as shown in FIGS. 15 and 16. Thus, the flexible dilator 26 has a tubular shape and is formed from a pliable polymer, such as a polyether block amide (e.g., PEBAX®) formed as a single integral piece along the length of the flexible dilator 26. In some examples, the interior surface of the flexible dilator 26 that surrounds the interior channel 28 (FIG. 5A) may include a hydrophilic lubricious coating or similar surface texture. Also, in some examples, the exterior surface of the flexible dilator 26 that slidably engages within the access sheath may include a hydrophilic lubricious coating or similar surface texture. The distal tip portion 26a of the flexible dilator 26, such as shown in FIG. 8, may include a tapered shape that protrudes from the distal end of the access sheath and operates to assist with insertion of the catheter system at the access site and advancement of the flexible dilator and catheter system in the vasculature.

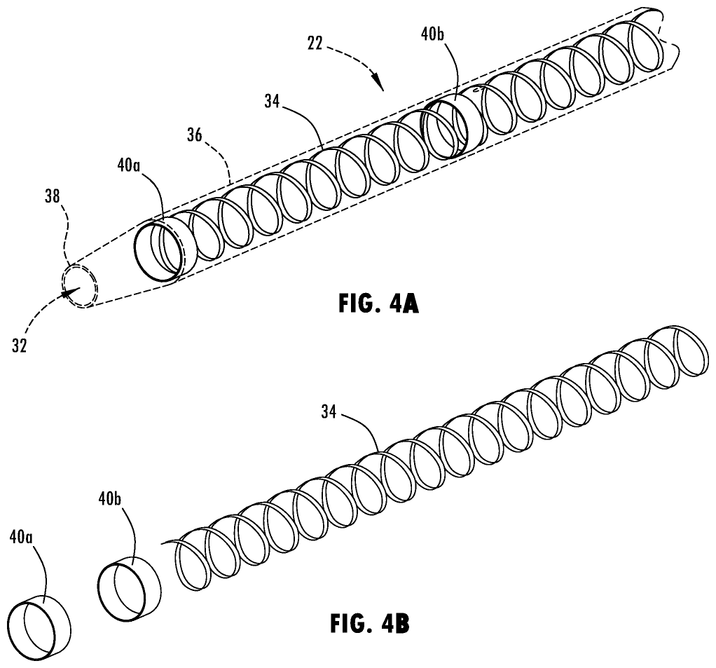

The access sheath 22, such as shown in FIG. 4A, has a tubular shape that defines or otherwise surrounds a central lumen 32. As illustrated in FIGS. 3A, 3B, and 4A, the central lumen 32 is sized to receive the flexible dilator 26 therein. The access sheath 22 is disposed over the flexible dilator 26 so as to be capable of simultaneously advancing with longitudinal movement of the flexible dilator 26 along an intravascular guide wire 30 (FIG. 7A). Once the access sheath 22 has advanced sufficiently in the vasculature, such as to a location that positions the reinforced intermediate section 24 at the tortuous section of the vasculature, the flexible dilator 26 is withdrawn from the central lumen 32 of the access sheath 22. With the dilator 26 removed, the central lumen 32 provides access to the location of the distal end 38 of the access sheath 22, such as to allow tools, such as diagnostic or interventional devices, to advance therein to exit the central lumen at or near a target treatment location.

In some examples, the access sheath 22 has an inner support structure 34 and an outer polymer 36 disposed over the inner support structure 34, such as illustrated at the distal end section 22a of the access sheath 22 shown in FIG. 4A. The inner support structure 34 is disposed around the central lumen 32 and extends at least partially along a length of the access sheath 22. The inner support structure 34 is configured to radially support the central lumen 32 and allow for longitudinal flexibility of access sheath 22, while generally maintaining the diameter of the hollow interior volume continuously along the length of the central lumen 32. The inner support structure 34 may be provided in various constructions and material types, such as rigid or semi-rigid materials that may be more or less rigid than the respective outer polymer 36. For example, the material of the support structure 34 may include a metal, such as stainless steel or other medical grade alloy, a flexible polymer, such as polyether block amide (e.g., PEBAX®), or combinations thereof. In some examples, the shape or construction of the inner support structure 34 of the access sheath 22 is provided as a coil (e.g., a ribbon coil or wire coil), a braiding, and/or a lattice structure. As shown for example in FIGS. 4A and 4B, the inner support structure 34 includes a stainless steel coil having a helical shape with a constant wrap angle along the axis of the central lumen 32. In other examples, the inner support structure may include a construction type or material that varies along the length, such as a construction that provides more a more rigid structure at the intermediate section of the access sheath, such as coil with a variable wrap angle, such as a tighter wrapping at the intermediate section of the access sheath. In some examples, inner support structure 34 includes a coil having constant or varying wrap angle and a substantially rectangular helical shape, a substantially elliptical helical shape, a substantially circular helical shape, or any combination thereof.

To locate the access sheath 22 as it is advanced in the vasculature, radiographic imaging may be used to locate a distal end 38 of the access sheath 22, such as to locate the distal end 38 at or near a desired location. For example, as shown in FIGS. 4A and 4B, two radiopaque markers 40a, 40b are disposed at the distal end section 22a of the access sheath. The radiopaque markers 40a, 40b are disposed over the coil shaped inner support structure 32, or differently stated, are disposed radially outside of the coil. In additional examples, the radiopaque makers may be disposed radially inside the coil or at the same radial location, such as an integrated piece or portion of the coil. The radiopaque marker or markers may be provided in various shapes and configurations and different types of radiopaque materials, such as to provide different shaped, sized, or colored indicators at different sections of the access sheath. As shown, for example, in FIG. 4A, the radiopaque markers 40a, 40b are bands that include a radiopaque material, for example platinum, tantalum, tungsten, palladium, and/or iridium. Other radiopaque materials are also possible. In some embodiments, a material may be considered radiopaque, for example, if the average atomic number is greater than 24 or if the density is greater than about 9.9 g/cm³. In some embodiments a distal portion of the access sheath may be infused with a radiopaque material so that the entire distal portion is visible using imaging techniques, and in other embodiments.

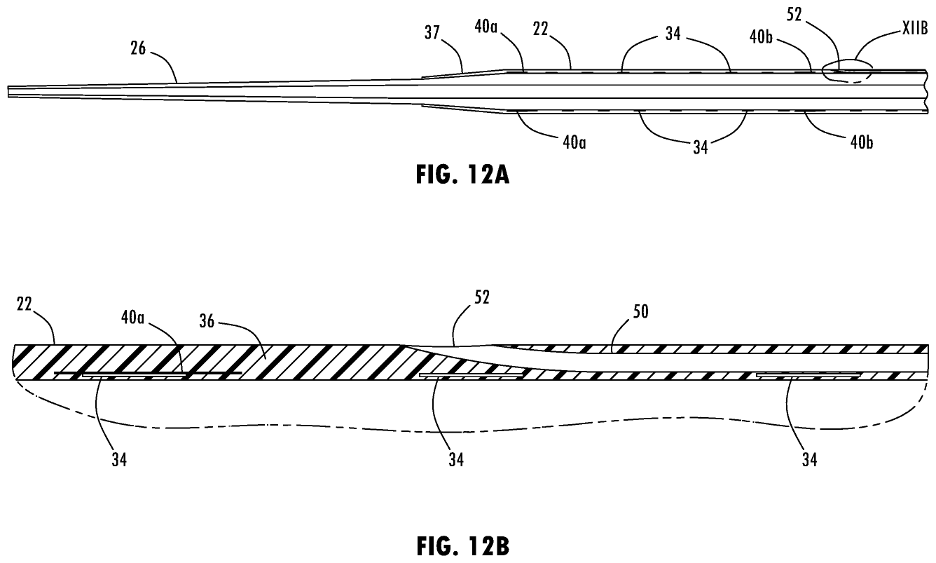

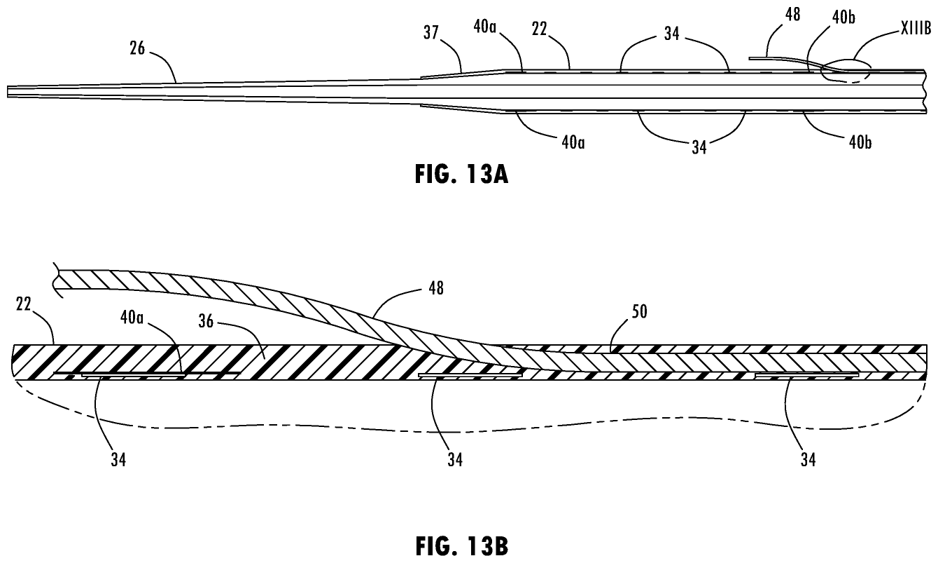

The distal end section 22a of the access sheath 22, such as shown in FIG. 5A, may also include a tapered tip 37 that forms the distal tip 38. The tapered tip 37 may be a separate piece of polymer material or may be integrally formed with the outer polymer 36 of the access sheath. As shown in FIGS. 12A and 13A, the tapered shaped of the tapered tip 37 provides a reduced opening size that mates with a corresponding tapered shape of the distal tip portion 26a of the flexible dilator 26, such as to prevent the flexible dilator from being advanced beyond the inserted position shown in FIGS. 3A and 3B.

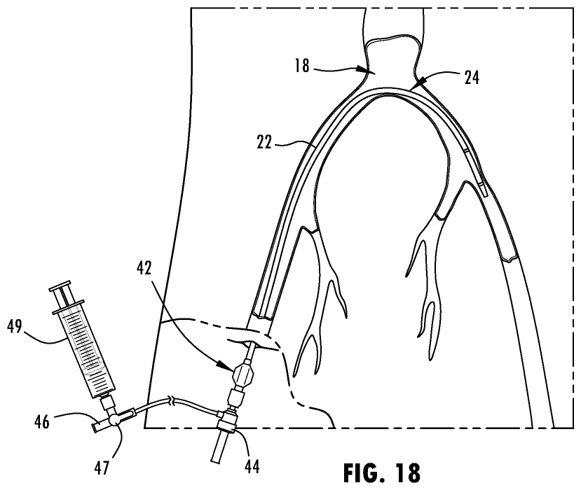

The proximal end of the access sheath 22 may include a hemostasis valve 42 (or an adapter for attaching the access sheath to a hemostasis valve) that prevents blood reflux when the catheter assembly 20 is inserted in a vessel and the valve 42 is used to maintain hemostasis during use of diagnostic or interventional devices with the access sheath 22. As shown in FIGS. 3A and 3B, the hemostasis valve 42 includes an axial port 44 and a cross-cut port 46. The axial port 44 may be used to receive the flexible dilator 26 and/or the guide wire 30 for advancing the access sheath 22 in the vasculature. Also, the axial port 44 may maintain a seal around diagnostic/interventional devices during procedures. The cross-cut port 46 may be used to administer fluids (e.g., contrast agents, heparin, saline, or any combination thereof), to receive or introduce a secondary wire 48, such as shown in FIG. 3A, to aspirate fluids from the catheter system and/or the patient, or any combination thereof. In some examples, the cross-cut port 46 may comprise threads or other engagement feature for connecting to a syringe 49 (FIG. 18) for aspirating a fluid (e.g., blood) or for administering a fluid to the patient. In some examples, the cross-cut port may comprise a valve 47 (e.g., a stopcock or the like) for controlling the administration or aspiration of fluids through the catheter system. And, in some examples, the hemostasis valve may include more or fewer ports and may be differently shaped and arranged from the illustrated example provided herein.

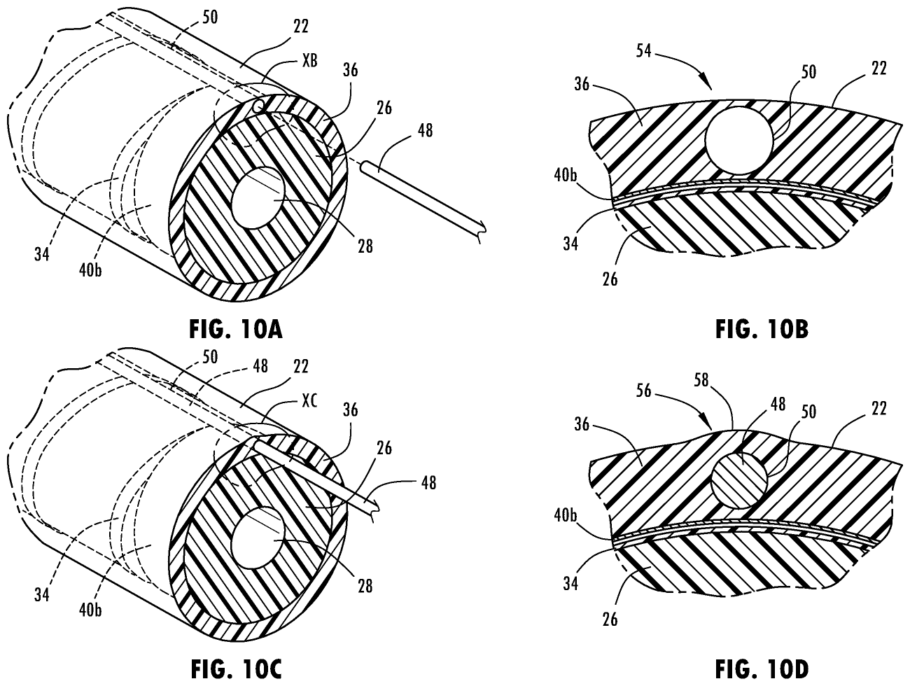

The outer polymer 36 of the access sheath 22 is generally disposed over the inner support structure 34 and extends from the distal end section 22a to the proximal end section 22b, such as shown in FIGS. 3A and 3B extending between the distal end 38 and the hemostasis valve 42 (or adapter for such valve). As shown in FIG. 4A, the outer polymer 36 encases the inner support structure 34 and the radiopaque markers 40a, 40b, such that the exterior surface of the outer polymer 36 is disposed radially away from the radiopaque markers 40a, 40b and the inner support structure 34. The outer polymer 36 may also extend radially inward from the inner support structure 34 and the radiopaque markers 40a, 40b, such as shown FIG. 5B. As such the outer polymer 36 shown in FIG. 5A, surrounds the inner support structure 34 and the radiopaque markers 40a, 40b, such as to cover the radially exterior and interior surfaces thereof (FIG. 10B). Thus, in the example shown in FIG. 5A, in addition to providing the exterior surface of the access sheath 22, the exterior polymer 36 also defines the interior surface that extends along the central lumen 32 (FIG. 4A) of the access sheath 22. In some examples, the exterior surface and/or interior surface of the access sheath may include a hydrophilic lubricious coating or similar surface texture, for example a coating having a low friction coefficient, to advantageously allow for smoother navigation through tortuous vasculature. In some implementations, the catheter coating has anti-thrombotic properties to advantageously inhibit thrombus formation. Further, in some examples, a separate substrate or material, such as a different polymer or separately formed polymer, may be disposed radially inward form the inner support structure. For example, the access sheath may also have an inner polymer that defines a lubricious inner surface of the central lumen.

The exterior surface of the outer polymer 36 (FIG. 5A) defines a generally consistent outer diameter of the access sheath 22 that extends along the length of the access sheath 22, such as shown in FIGS. 3A and 3B. In some examples, the size (i.e., outer diameter) of the access sheath 22 is 4 French, 5 French, 6 French, 7 French, or 8 French. In some implementations, such as for use at a femoral access 10 (FIG. 1), the outer diameter of the access sheath 22 is between about 5 French and about 8 French (between about 1.7 mm and about 2.7 mm). For example, the access sheath used at the femoral access 10 may preferably have an outer diameter of about 5 French or about 6 French. In some implementations, such as for use at a radial access 12 (FIG. 1), the outer diameter of the access sheath is between about 4 French and about 6 French (e.g., between about 1.3 mm and about 2 mm). In yet other implementations, such as for use at a pedal access 16 (FIG. 1), the outer diameter of the access sheath is between about 4 French and about 6 French (e.g., between about 1.3 mm and about 2 mm). Other sizes are also possible, for example depending on the size of the target body lumen of a particular patient.

In some embodiments, the access sheath 22 has a length of about 45 cm to about 135 cm (e.g., between about 45 cm and about 135 cm), such as generally standard lengths of about 45 cm, about 60 cm, about 90 cm, and about 135 cm. For instance, such as in femoral access procedures, the access sheath 22 has a length of about 45 cm. However, other lengths are also possible, for example to allow for insertion of the access sheath at other access sites in the femoral, radial, brachial, or subclavian artery.

The interior surface of the central lumen 32 (FIG. 4A) of the access sheath 22 shown in FIGS. 3A and 3B defines a generally consistent inner diameter of the access sheath 22 that extends along the length of the access sheath 22. In some examples, the inner diameter of the access sheath 22 is about 4 French, about 5 French, or about 6 French. In some implementations, such as for use at a femoral access 10 (FIG. 1), the inner diameter of the access sheath 22 is between about 3 French and about 6 French (between about 1 mm and about 2 mm). With such a consistent inner diameter and a consistent outer diameter, the wall thickness of the access sheath is also generally consistent along the length of the access sheath. The wall thickness may be defined between the interior surface and the exterior surface of the access sheath, such as shown in FIG. 5B.

The outer polymer 36 of the access sheath 22 may be formed as a single piece, such as via extrusion, injection molding, or another suitable process. The outer polymer 36 is may be a flexible polymer material, such with polyether block amide (e.g., PEBAX®) formed as a single type of material along the length of the access sheath. In some examples, the outer polymer may formed from a polyether block amide, polyurethane, silicone, latex, polytetrafluoroethylene (PTFE), a plastic material, any combination thereof, or the like.

To provide a reinforcement feature at the intermediate section 24 of the access sheath 20, the outer polymer 36 includes an increased durometer at the intermediate section 24 relative to the distal and proximal end sections of the access sheath 20. The increased durometer provides a relatively more stiff or rigid area at the intermediate section 24, which functions to reduce the risk of kinking the access sheath at this section due to longitudinal bending, such as result of traversing tortuously curves in the vasculature. Likewise, the reduced durometer at the distal end section provides ease in initially conforming the access sheath to the shape and curvature of the vasculature upon advancement. Further, the reduced durometer at the proximal end section can provide shape conformity of the access sheath at the access site.

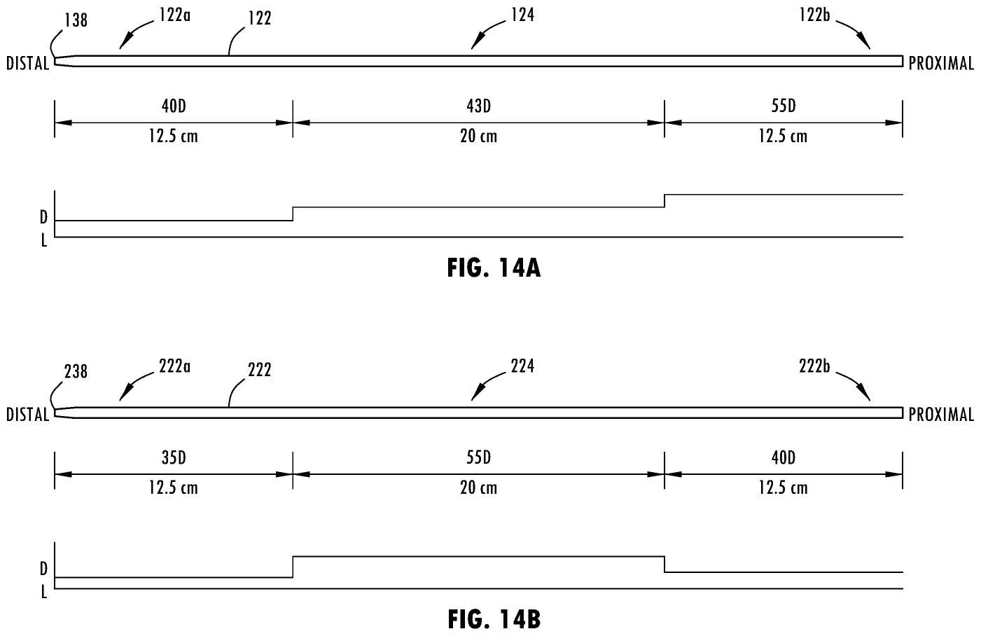

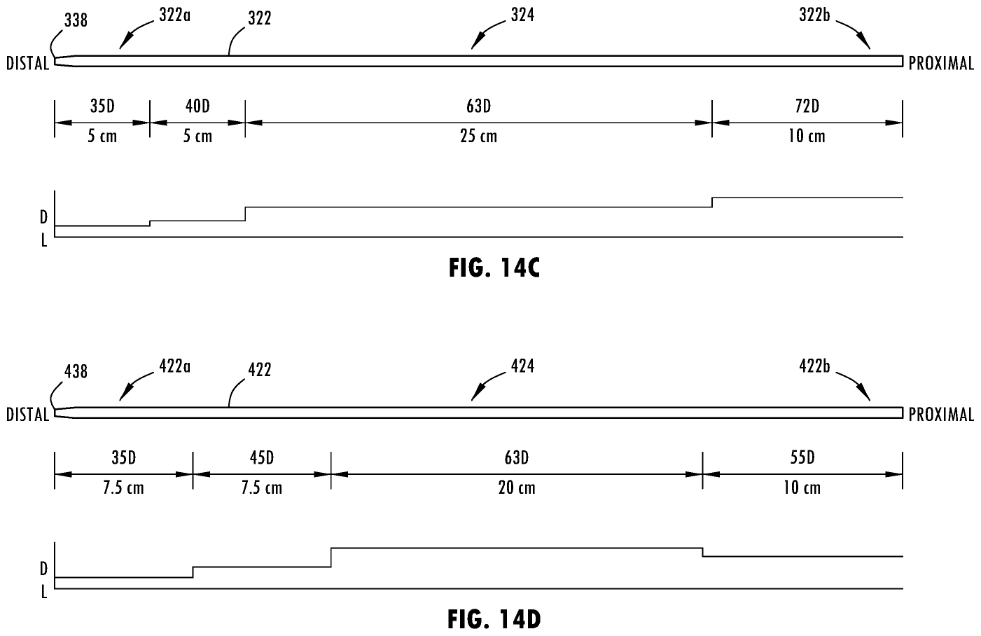

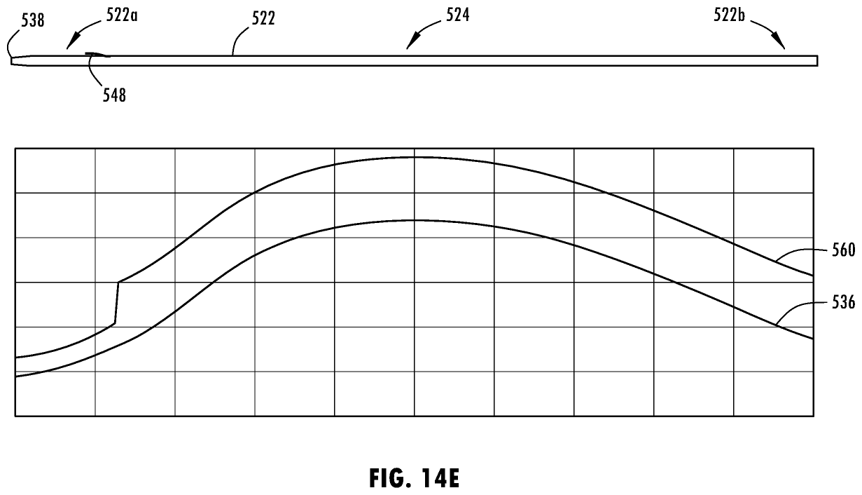

In some examples, the outer polymer 36 has a different material composition or material state at the distal end section 22a and the proximal end section 22b that provides a durometer that is less that the durometer provided by the material composition or state of the outer polymer 36 at the intermediate section 24. The increased durometer at the intermediate section 24 is a property of the polymer material that is independent of the wall thickness of the access sheath, as the outer diameter, inner diameter, and corresponding wall thickness are generally constant along the length and across the durometer transitions along the length of the access sheath. For example, the outer polymer 36 at the intermediate section 24 may include a durometer of at least 2 Shore D hardness greater than the durometer at least the distal end section 22a. In other examples, the intermediate section 24 may include a durometer of at least 5 Shore D hardness greater than the durometer at the distal and proximal end sections 22a, 22b. The changes in durometer along the length of the access sheath may be stark between discrete sections, such as shown in FIGS. 14A-14D, or may be gradual transitions between defined or undefined sections, such as shown in FIG. 14E. For example, in the case of relatively defined sections having a constant durometer, the access sheath has at least three sections and may include more defined sections, such as four, five, or more sections.

As shown for example in FIG. 14A, an access sheath 122 has three discrete sections that provide different durometer values. The intermediate section 124 is about 24 cm in length and has a durometer of about 43 D on the Shore D hardness scale. The distal end section 122a is about 12.5 cm in length from the distal end 138 to the intermediate section 124 and has a durometer of about 40 D on the Shore D hardness scale. The proximal end section 122b is also about 12.5 cm in length from the intermediate section 124 to the proximal end, but instead has a durometer of about 55 D on the Shore D hardness scale. Accordingly, it is contemplated that in some examples that the proximal end section may have the same or a greater durometer as the intermediate section. These durometer values are shown in the corresponding chart in FIG. 14A, illustrating that there is a stark transition in durometers between the discrete sections of the access sheath.

Another example shown in FIG. 14B provides an access sheath 222 that also has three discrete sections, each with different durometer values. The intermediate section 224 is about 20 cm in length and has a durometer of about 55 D on the Shore D hardness scale. The distal end section 222a is about 12.5 cm in length from the distal end 238 to the intermediate section 224 and has a durometer of about 35 D on the Shore D hardness scale. The proximal end section 222b is also about 12.5 cm in length from the intermediate section 224 to the proximal end and has a durometer of 40 D on the Shore D hardness scale. These durometer values are shown in the corresponding chart in FIG. 14B, illustrating that there is a stark transition in durometers between the discrete sections of the access sheath.

As further shown in FIG. 14C, another example of an access sheath 322 has four discrete sections that provide different durometer values. The intermediate section 324 is about 25 cm in length and has a durometer of about 63 D on the Shore D hardness scale. The distal end section 322a is about 10 cm in length and sub-divided into two equal sections of about 5 cm each having a durometer of about 35 D and 40 D on the Shore D hardness scale. The proximal end section 322b is also about 10 cm in length from the intermediate section 324 to the proximal end and has a durometer of 72 D on the Shore D hardness scale. These durometer values are shown in the corresponding chart in FIG. 14C, illustrating that there is a stark transition in durometers between the discrete sections of the access sheath.

As shown in a further example in FIG. 14D, an access sheath 422 also has four discrete sections that provide different durometer values. The intermediate section 424 is about 20 cm in length and has a durometer of about 63 D on the Shore D hardness scale. The distal end section 422a is about 15 cm in length and sub-divided into two equal sections of about 7.5 cm each having a durometer of about 35 D and 45 D on the Shore D hardness scale. The proximal end section 422b is also about 10 cm in length from the intermediate section 424 to the proximal end and has a durometer of 55 D on the Shore D hardness scale. These durometer values are shown in the corresponding chart in FIG. 14D, illustrating that there is a stark transition in durometers between the discrete sections of the access sheath.

With respect to the example shown in FIG. 14E, the durometer chart of the outer polymer 536 of the corresponding access sheath 522 has a gradual durometer transition along the length between undefined sections, shown as a generally parabolic shape at the intermediate section 524 with flattening end sections 522a, 522b. As shown in FIG. 14E, the distal end 538 has a durometer of approximately 35 D on the Shore D hardness scale, the proximal end has a durometer of approximately 40 D on the Shore D hardness scale, and the intermediate section peaks with a durometer of approximately 55 D on the Shore D hardness scale.

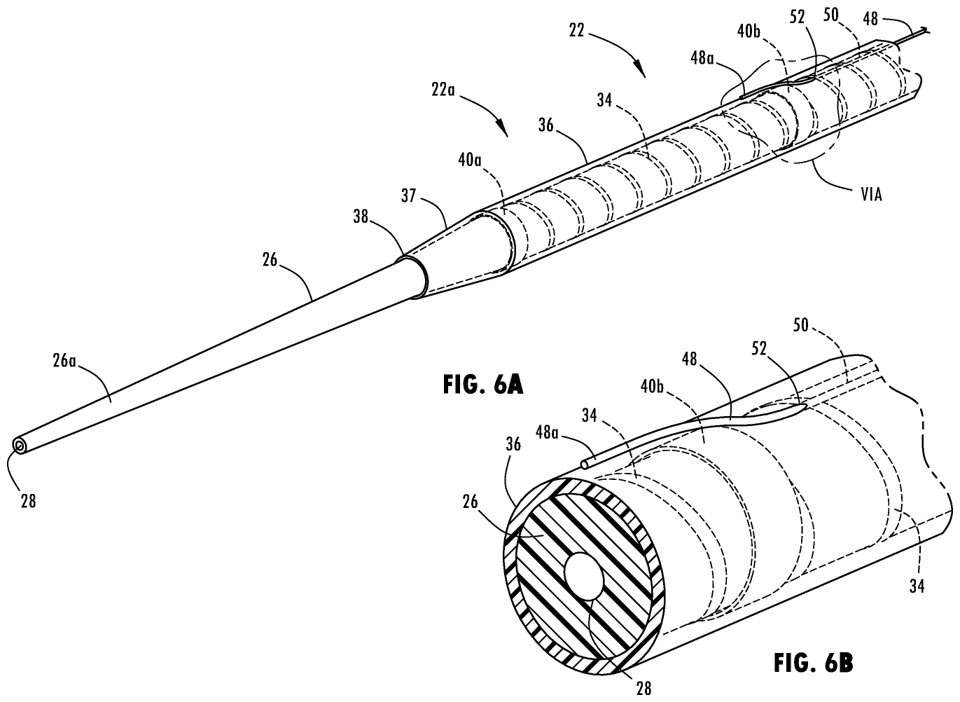

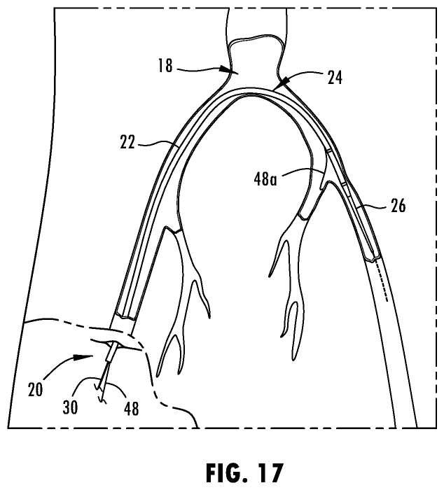

Referring again to FIGS. 3A-13B, another reinforcement feature that may be provided at the intermediate section 24 of the access sheath 20 is an accessory or secondary wire 48 that is inserted along a secondary or auxiliary lumen 50 disposed in the wall thickness of the access sheath 22. The secondary wire 48 provides reinforcement and increased durometer at the intermediate section, such as shown in line 560 in FIG. 14E that accounts for the durometer of the exterior polymer and the secondary wire. Similar to increased durometer, the secondary wire makes the intermediate section more resistant to kinking at the intermediate section when the flexible dilator is removed from the central lumen of the access sheath. Thus, the secondary wire increases the stiffness of the access sheath to prevent the intermediate section of the access sheath from kinking. Further, in some examples, the auxiliary wire may also be used for anchoring the access sheath near a target site or treatment location, such as shown in FIG. 17.

As shown in FIGS. 5A and 5B, the outer polymer 36 includes a secondary lumen 50 disposed between the exterior surface and the central lumen 32. The secondary lumen 50 extends at least partially along the length of the access sheath 22, such as extending along the intermediate section 24 desired to be reinforced for kink prevention. As shown in FIGS. 12A and 12B, the secondary lumen 32 is generally linear disposed along the access sheath 22. In additional examples the secondary lumen may be disposed helically along the access sheath. As further shown in FIGS. 10B and 12B, the secondary lumen 32 is disposed in the exterior polymer 36 radially outside of the interior support structure 34. However, in additional examples, the secondary lumen may be disposed radially inside or otherwise at the generally same level as the interior support structure of the access sheath, such as in the example of a helical secondary lumen that follows a coil shape of the interior support structure.

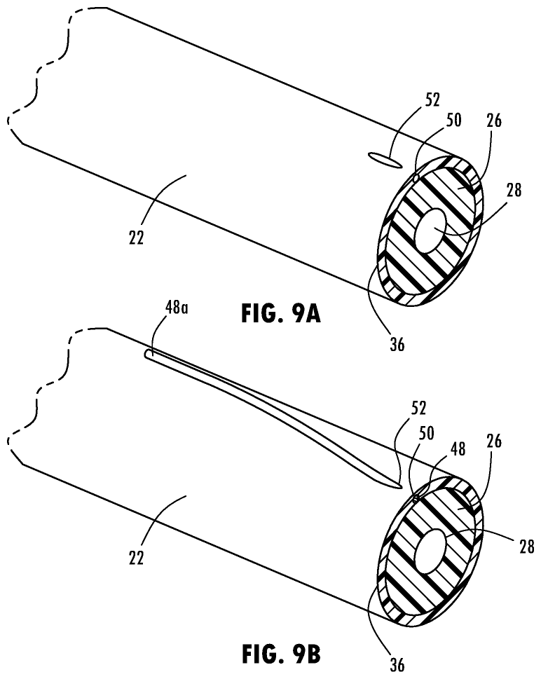

The secondary lumen 50 has an exit opening 52 at the exterior surface of the outer polymer at a longitudinal location proximal to the distal tip 38 of the access sheath 22. The exit opening 52 may be at the exterior surface of the outer polymer 38 at a location proximal to the distal tip 38 of the access sheath 22, such as between about 1 cm and 5 cm (e.g., between about 2 cm and 4 cm) from the distal tip of the access sheath for the exit opening to align with peripheral vessels in contralateral femoral access procedures. As shown in FIGS. 9A and 12B, the exit opening 52 has an elongated shape that is elongated in the longitudinal dimension of the access sheath 22. The elongated shape of the exit opening may provide a larger diameter than the secondary wire, such as to provide flexibility in maneuvering the distal portion of secondary wire that exits the exit opening.

In some examples, such as shown in FIGS. 5A-7A, a radiopaque marker 40b is disposed at or near the exit opening 52 for positioning the exit opening 52 at a desired location in a patient's anatomy. The secondary wire 48 is inserted along the secondary lumen 50, such that when the flexible dilator is removed from the central lumen 32 of the access sheath 22, the secondary wire 48 is configured to prevent the intermediate section of the access sheath from kinking. In some examples, the secondary lumen 50 may also or alternatively be used to dispense drugs and/or contrast agents through the lumen. Further examples may also provide additional secondary lumens in the outer polymer of the access sheath, such as at opposing sides of the access sheath.

In some examples of the secondary wire, such as shown in FIG. 8, the distal end 48a of the secondary wire 48 has a blunted tip and a hydrophilic coating that is configured to reduce friction when entering and advancing through the secondary lumen 50 and the vasculature. And, in some embodiments, the distal end 48a of the secondary wire 48 may comprise a radiopaque material. The secondary wire 48 may have a diameter between about 0.014 inches to about 0.035 inches, such as the standard diameters of about 0.014 inches, about 0.018 inches, or about 0.035 inches. However, other diameters may be used. Also, the secondary wire may be made of nitinol, stainless steel, carbon fiber, or the like and may be inserted into the secondary lumen with a wire introducer. Further, the secondary wire may comprise a preformed shape, such as a memory shape, that is used to manage the vasculature at the intermediate section and/or used to provide shapes useful in intravascular procedures or anchoring, such as at the distal tip that protrudes from the exit opening.

In some examples, as shown in FIG. 3A, the secondary lumen 50 has an entrance opening that is engaged with a valve port 46 of the hemostasis valve 42, such that the valve port 46 receives the secondary wire 48 via an introducer device. In some examples, the entrance opening of the secondary lumen has a port (e.g., a valve port) that is separate from the hemostasis valve, such as to provide separation for the surgeon accessing and manipulating the wire and access sheath.

In some examples, as shown in FIG. 3B, the secondary lumen 50 has an entrance opening in the access sheath 22 that is distal to the homeostasis valve 42. For instance, the secondary lumen 50 comprises an entrance opening having a port (e.g., valve port) that is engaged with an introducer device (not shown) that is distal to the homeostasis valve 42. In other examples, the entrance opening of the secondary lumen engages with a port (e.g., valve port) at a junction (e.g., adapter) of the homeostasis valve and the access sheath using an optional introducer device.

Referring now to FIGS. 10A-10D, the secondary lumen 50, in some implementations, assumes a compressed or contracted state 54 (FIG. 10B) when the secondary wire 48 is not present in the secondary lumen 50. For example, in the compressed or contracted state 54 the secondary lumen 50 may be substantially closed or collapsed, at least at the exit opening, so as to prevent fluid from entering the secondary lumen and to maintain a substantially consistent outer circumference along the exterior surface of the access sheath 22, such as when the access sheath 22 is inserted at the access site. As shown in FIG. 10D, when the secondary wire 48 is inserted and present in the secondary lumen 50, the secondary lumen 50 assumes an expanded state 56. As shown in FIG. 10C, the insertion of the secondary wire 48 causes the circular shaped secondary lumen 50 (FIG. 10B) to expand in diameter from its initial diameter in the contracted state. This expansion to the expanded state 56, in some examples, may form a raised area or ridge 58 along the exterior surface of the access sheath 22, such as shown in FIG. 10D. The expansion of the secondary lumen is provided by the flexibility of exterior polymer 36. However, in some examples, the secondary lumen may have the same or larger diameter than the secondary wire, such that no expansion occurs when the secondary wire is inserted.

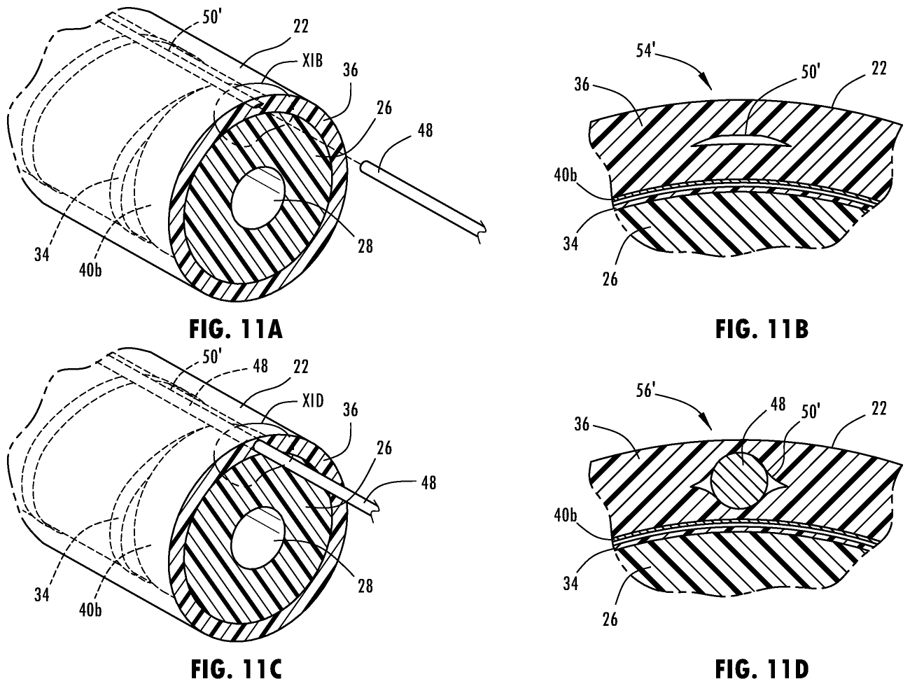

In some examples, the secondary lumen in the contracted state provides a cross-sectional shape that is a slotted shape, an ovular shape, a circular shape, or a crescent shape. For example, as shown in FIGS. 11A-11D, the secondary lumen 50′ has a crescent shape in the contracted state 54′ (FIG. 11B). As shown in FIG. 11C, the insertion of the secondary wire 48 causes the secondary lumen 50′ (FIG. 11B) to expand from its shape in the contracted state to provide sufficient volume for the secondary wire. This expansion to the expanded state 56′ shown in FIG. 11D does not form a raised area or ridge along the exterior surface of the access sheath 22, as the shape and material surrounding the secondary lumen 501 is configured to generally prevent alterations to the exterior surface, so as to maintain the French size of the access sheath.

Referring now to FIGS. 15-18, an example of a procedural method that utilizing an access sheath as disclosed herein is illustrated. As shown in FIG. 15, initially a guide wire 30 is inserted is through the patient's skin at a contralateral percutaneous femoral access 10 through an aortic bifurcation 18 and into a common femoral artery (CFA). A catheter assembly 20 is advanced over the guide wire 30, such as through the aortic bifurcation 18 and into the CFA toward a target site or treatment location. As shown in FIGS. 15 and 16, the flexible dilator 26 carrying the access sheath 22 is first inserted through the skin at the access site 10 along the guide wire 30. The access sheath 22 disposed over the flexible dilator 26 is simultaneously advanced with the flexible dilator 26 along the intravascular guide wire and over the aortic bifurcation 18. After passing the aortic bifurcation, the surgeon may monitor the location of the distal end of the access sheath 22 by viewing the radiopaque markers 40a, 40b, such as with X-ray or other medical imaging devices. As such, the radiopaque marker 40b near the exit opening of the secondary lumen may be positioned adjacent or nearly adjacent to a peripheral vessel that can be used as an anchoring location for a secondary wire.

As shown in FIG. 17, the secondary wire 48 is inserted through the secondary lumen to the exit opening. The secondary wire 48 may exit the exit opening and anchor at the peripheral vessel shown in FIG. 17 or any other desired anchoring anatomy for securing the access sheath near the target treatment location. With the secondary wire 48 inserted, the flexible dilator 26 is withdrawn from the access sheath 22 to expose the central lumen for a tool or other surgical device to access the target treatment location. When the flexible dilator 26 is removed from the central lumen of the access sheath 22, such as shown in FIG. 18, the intermediate section 24 of the access sheath 22 that spans the aortic bifurcation is prevented from collapsing or kinking because a durometer of the outer polymer at the intermediate section exceeds a threshold hardness and the secondary wire provides additional support. In some instances (not shown), the secondary wire may only be partially inserted into the secondary lumen (i.e., the distal tip 48a of the secondary wire remains entirely within the secondary lumen) to provide anti-kinking support to the access sheath depending on the demands of the patient's vasculature.

As used herein, a “body lumen” refers to the inside space of a tubular structure in the body, such as an artery, intestine, vein, gastrointestinal tract, bronchi, renal tubules, and urinary collecting ducts. In some instances, a body lumen refers to the aorta.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

It is to be understood by one having ordinary skill in the art that the specific devices and processes illustrated in the attached drawings and described in this specification are simply example embodiments of the inventive concepts defined in the appended claims. Hence, specific dimensions and other physical characteristics relating to the embodiments disclosed herein are not to be considered as limiting, unless the claims expressly state otherwise. It is also to be understood that construction of the described invention and other components is not limited to any specific material. Other example embodiments of the invention disclosed herein may be formed from a wide variety of materials, unless described otherwise herein.

Changes and modifications in the specifically-described embodiments may be carried out without departing from the principles of the present invention, which is intended to be limited only by the scope of the appended claims as interpreted according to the principles of patent law including the doctrine of equivalents.

What is claimed is:

1. A vascular catheter system comprising:
a flexible dilator having an interior channel configured to slidably engage an intravascular guide wire that leads toward a target treatment location in a patient's anatomy;
an access sheath having a tubular shape that defines a central lumen, the access sheath configured to be disposed over the flexible dilator so as to simultaneously advance with the flexible dilator along an intravascular guide wire toward a target treatment location, the access sheath comprising:
an inner support structure disposed around the central lumen and extending at least partially along a length of the access sheath;
a radiopaque marker disposed at a distal end section of the access sheath;
a hemostasis valve disposed at a proximal end section of the access sheath;
an outer polymer encasing the inner support structure and the radiopaque marker, the outer polymer having an exterior surface extending between the radiopaque marker and the hemostasis valve that defines a consistent outer diameter; and
wherein the outer polymer comprises a first durometer at the proximal end section of the access sheath, a second durometer at an intermediate section of the access sheath, and a third durometer at the proximal end section of the access sheath, the second durometer being greater than the first and third durometers;
wherein the flexible dilator is configured to be withdrawn from the central lumen of the access sheath to provide tool access to a target treatment location, and
wherein when the flexible dilator is removed from the central lumen of the access sheath, the second durometer of the outer polymer is configured to prevent the intermediate section of the access sheath from collapsing or kinking.

2. The vascular catheter system of claim 1, wherein the outer polymer comprises a secondary lumen disposed between the exterior surface and the central lumen, the secondary lumen extending at least partially along the length of the access sheath.

3. The vascular catheter system of claim 2, wherein the secondary lumen comprises an exit opening at the exterior surface of the outer polymer at a longitudinal location proximal to a distal tip of the access sheath.

4. The vascular catheter system of claim 3, wherein the radiopaque marker is disposed at or near the exit opening for positioning the exit opening at a desired location in a patient's anatomy.

5. The vascular catheter system of claim 3, further comprising a secondary wire configured to be inserted along the secondary lumen to the exit opening, wherein when the flexible dilator is removed from the central lumen of the access sheath, the secondary wire is configured to prevent the intermediate section of the access sheath from kinking.

6. The vascular catheter system of claim 5, wherein secondary wire comprises a blunted tip and a hydrophilic coating configured to reduce friction when advancing through the secondary lumen.

7. The vascular catheter system of claim 5, wherein the secondary lumen comprises an entrance opening engaged with a valve port of the hemostasis valve, the valve port configured to receive the secondary wire via an introducer device and direct the secondary wire into the secondary lumen.

8. The vascular catheter system of claim 5, wherein the secondary lumen comprises a contracted state defined by the secondary wire not being present in the secondary lumen and the secondary lumen having an initial diameter, and wherein the secondary lumen comprises an expanded state defined by the secondary wire being present in and expanding the secondary lumen to a second diameter that is greater than the initial diameter.

9. The vascular catheter system of claim 8, wherein with the secondary lumen in the expanded state, the exterior surface of the access sheath includes a longitudinal ridge defined by the expanded secondary lumen.

10. The vascular catheter system of claim 1, wherein with the access sheath comprises a distal tip having a tapered shape.

11. The vascular catheter system of claim 1, wherein with the inner support structure comprises a coil or a braiding with longitudinal flexibility.

12. The vascular catheter system of claim 1, wherein with the inner support structure comprises a stainless steel coil or a stainless steel braiding.

13. The vascular catheter system of claim 1, wherein the outer polymer comprises polyether block amide formed as a single integral piece along the length of the access sheath.

14. The vascular catheter system of claim 1, wherein the access sheath comprises an inner polymer that defines a lubricious inner surface of the central lumen.

15. The vascular catheter system of claim 14 wherein the inner polymer comprises polyether block amide formed as a single integral piece with the outer polymer along the length of the access sheath.

16. The vascular catheter system of claim 14, wherein a wall thickness of the access sheath is defined between the inner surface and the outer surface, and wherein the wall thickness is substantially consistent along the length of the access sheath.

17. The vascular catheter system of claim 1, wherein the consistent outer diameter of the access sheath comprises 4 French, 5 French, 6 French, 7 French, or 8 French.

18. The vascular catheter system of claim 1, wherein an inner diameter of the central lumen of the access sheath is substantially constant along the length of the access sheath and comprises 4 French, 5 French, or 6 French.

19. The vascular catheter system of claim 1, wherein the secondary wire comprises a diameter of 0.018 or 0.035 inches.

20. The vascular catheter system of claim 1, wherein the length of the access sheath is between 35 cm and 55 cm.

21. The vascular catheter system of claim 1, wherein the first durometer is disposed along at least 7 cm of the length of the access sheath, wherein the second durometer is disposed along at least 20 cm of the length of the access sheath, and wherein the third durometer is disposed along at least 7 cm of the length of the access sheath.

22. A vascular access sheath comprising:
a hemostasis valve disposed at a proximal end section the access sheath;
an inner support structure extending from the hemostasis valve toward a distal end section of the access sheath, the inner support structure defining a tubular shape that extends around a central lumen of the access sheath;
an outer polymer disposed over the inner support structure, the outer polymer having an exterior surface extending from the hemostasis valve to a distal tip of the access sheath;
wherein the outer polymer comprises a consistent thickness along the access sheath and defines a greater durometer an intermediate section of the access sheath than at the proximal and distal end sections of the access sheath; and
wherein the outer polymer comprises a secondary lumen disposed between the exterior surface and the central lumen, the secondary lumen extending at least partially along the length of the access sheath and configured to receive a wire to reinforce the stiffness of the intermediate section of the access sheath.

23. The vascular access sheath of claim 21, further comprising a radiopaque marker disposed at the distal end section of the access sheath.

24. The vascular access sheath of claim 21, further comprising a first radiopaque marker disposed at the distal tip the access sheath and a second radiopaque marker disposed at or near an exit opening of the secondary lumen.

25. The vascular access sheath of claim 21, wherein the secondary lumen comprises an exit opening at the exterior surface of the outer polymer at a location proximal to the distal tip of the access sheath.

26. The vascular access sheath of claim 25, wherein the exit opening of the secondary lumen is between 2 and 4 cm from the distal tip of the access sheath.

27. The vascular access sheath of claim 25, wherein the secondary lumen comprises an entrance opening engaged with a valve port of the hemostasis valve, the valve port configured to receive the secondary wire and direct the secondary wire into the secondary lumen.

28. The vascular access sheath of claim 21, wherein the secondary lumen comprises a contracted state defined by the secondary wire not being present in the secondary lumen and the secondary lumen having an initial diameter, and wherein the secondary lumen comprises an expanded state defined by the secondary wire being present in and expanding the secondary lumen to a second diameter that is greater than the initial diameter.

29. The vascular access sheath of claim 28, wherein with the secondary lumen in the expanded state, the exterior surface of the access sheath includes a longitudinal ridge defined by the expanded secondary lumen.

30. The vascular access sheath of claim 28, wherein with the secondary lumen in the contracted state, a cross-sectional shape of the secondary lumen comprises at least one of a slotted shape, an ovular shape, a circular shape, or a crescent shape.

31. The vascular access sheath of claim 21, wherein with the access sheath comprises a distal tip having a tapered shape.

32. The vascular access sheath of claim 21, wherein with the inner support structure comprises a metal coil or metal braiding.

33. The vascular access sheath of claim 21, wherein the outer polymer comprises polyether block amide formed as a single integral piece along the length of the access sheath.

34. The vascular access sheath of claim 21, wherein the access sheath comprises an inner polymer that defines a lubricious inner surface of the central lumen.

35. The vascular access sheath of claim 34 wherein the inner polymer comprises polyether block amide formed as a single integral piece with the outer polymer along the length of the access sheath.

36. The vascular access sheath of claim 34, wherein a wall thickness of the access sheath is defined between the inner surface and the outer surface, and wherein the wall thickness is substantially consistent along the length of the access sheath.

37. The vascular access sheath of claim 21, wherein the consistent outer diameter of the access sheath comprises 4 French, 5 French, 6 French, 7 French, or 8 French.

38. The vascular access sheath of claim 21, wherein an inner diameter of the central lumen of the access sheath is substantially constant along the length of the access sheath and comprises 4 French, 5 French, or 6 French.

39. The vascular access sheath of claim 21, wherein the length of the access sheath is between 35 cm and 55 cm.

40. The vascular access sheath of claim 21, wherein the first durometer is disposed along at least 7 cm of the length of the access sheath, wherein the second durometer is disposed along at least 20 cm of the length of the access sheath, and wherein the third durometer is disposed along at least 7 cm of the length of the access sheath.

41. A method for treating contralateral lower extremity vascular condition comprising:
advancing an intravascular guide wire from a contralateral percutaneous femoral access through an aortic bifurcation and into a common femoral artery (CFA);
advancing a catheter assembly over the guide wire and through the aortic bifurcation and into the CFA toward a target treatment location, the catheter assembly comprising:
an flexible dilator having an interior channel that surrounds the guide wire; and
an access sheath disposed over the flexible dilator so as to simultaneously advance with the flexible dilator along the intravascular guide wire, the access sheath comprising: (i) an inner support structure extending at least partially along a length of the access sheath, and (ii) an outer polymer encasing the inner support structure and defining an exterior surface that extends from a distal tip of the access sheath to a proximal end section of the access sheath;
withdrawing the flexible dilator from the access sheath to expose a central lumen of the access sheath configured for a tool to access the target treatment location;
wherein when the flexible dilator is removed from the central lumen of the access sheath, an intermediate section of the access sheath spanning the aortic bifurcation is prevented from collapsing or kinking because a durometer of the outer polymer at the intermediate section exceeds a threshold hardness; and
wherein the proximal end section and distal end section of the access sheath have durometers that are less than the durometer at the intermediate section to provide flexibility at the contralateral percutaneous femoral access and near the target treatment location.

42. The method of claim 41, further comprising
prior to removing the flexible dilator from the access sheath, inserting a secondary wire through a secondary lumen in the access sheath, the secondary lumen disposed longitudinally within a thickness of the outer polymer and having an exit opening at the exterior surface of the outer polymer proximal to the distal tip; and
wherein the secondary wire increases the stiffness of the access sheath to prevent the intermediate section of the access sheath from kinking.

43. The method of claim 42, wherein the access sheath comprises a consistent diameter and wall thickness along the length of the access sheath, wherein the secondary lumen comprises a contracted state defined by the secondary wire not being present in the secondary lumen, and wherein the secondary lumen comprises an expanded state defined by the secondary wire being present in and expanding the secondary lumen.

44. The method of claim 43, wherein with the secondary lumen in the expanded state, the exterior surface of the access sheath includes a longitudinal ridge defined by the expanded secondary lumen.

45. The method of claim 42, wherein the secondary wire comprises a diameter of 0.018 or 0.035 inches.

46. The method of claim 41, further comprising
positioning the access sheath in the vasculature using radiographic imaging to locate an exit opening of a secondary lumen in the access sheath near a desired anchoring anatomy, the secondary lumen disposed longitudinally within a thickness of the outer polymer and having the exit opening at the exterior surface of the outer polymer proximal to the distal tip;
prior to removing the flexible dilator from the access sheath, inserting a secondary wire through the secondary lumen to the exit opening; and
anchoring the secondary wire at the desired anchoring anatomy for securing the access sheath near the target treatment location.

47. The method of claim 46, wherein the access sheath comprises a radiopaque marker disposed at a distal end section of the access sheath near the exit opening.

48. The method of claim 46, wherein the access sheath comprises a hemostasis valve disposed at a proximal end section the access sheath, wherein the secondary lumen comprises an entrance opening engaged with a valve port of the hemostasis valve, and wherein the valve port is configured to receive the secondary wire via an introducer device and direct the secondary wire into the secondary lumen.

49. The method of claim 41, wherein with the inner support structure comprises a coil or a braiding.

50. The method of claim 41, wherein the outer polymer comprises polyether block amide formed as a single integral piece along the length of the access sheath.

51. The method of claim 41, wherein the access sheath comprises an inner polymer that defines a lubricious inner surface of the central lumen.

52. The method of claim 41, wherein the consistent outer diameter of the access sheath comprises 4 French, 5 French, 6 French, 7 French, or 8 French.

53. The method of claim 41, wherein an inner diameter of the central lumen of the access sheath is substantially constant along the length of the access sheath and comprises 4 French, 5 French, or 6 French.

54. The method of claim 41, wherein the length of the access sheath is between 35 cm and 55 cm.

55. The method of claim 41, wherein the first durometer is disposed along at least 7 cm of the length of the access sheath, wherein the second durometer is disposed along at least 20 cm of the length of the access sheath, and wherein the third durometer is disposed along at least 7 cm of the length of the access sheath.
